(19) United States
(12) Patent Application Publication
(10) Pub. No.: US 2004/0127978 A1
(43) Pub. Date: Jul. 1, 2004

(54) Active agent delivery system including a hydrophilic polymer, medical device, and method

(75) Inventors: Randall V. Sparer, Andover, MN (US); Christopher M. Hobot, Tonka Bay, MN (US); SuPing Lyu, Maple Grove, MN (US); Kiem Dang, Blaine, MN (US)

Correspondence Address: MUETING, RAASCH & GEBHARDT, P.A., P.O. BOX 581415, MINNEAPOLIS, MN, 55458, US

(73) Assignee: Medtronic, Inc., Minneapolis, MN

(21) Appl. No.: 10/640,713

(22) Filed: Aug. 13, 2003

Related U.S. Application Data
(60) Provisional application No. 60/403,392, filed on Aug. 13, 2002.

(51) Int. Cl. A61F 2/06

(52) U.S. Cl. 623/1.46

(57) ABSTRACT

The present invention provides active agent delivery systems for use in medical devices, wherein the active agent delivery systems include an active agent and a hydrophilic miscible polymer blend that includes an active agent and a miscible polymer blend comprising a hydrophilic polymer (preferably, a polyurethane) and a second polymer having a different swellability in water at 37° C.

### CROSS-REFERENCE TO RELATED APPLICATIONS

The present application claims priority to U.S. Provisional Patent Application Serial No. 60/403,392, filed on Aug. 13, 2002, which is incorporated herein by reference in its entirety.

### BACKGROUND OF THE INVENTION

A polymeric coating on a medical device may serve as a repository for delivery of an active agent (e.g., a therapeutic agent) to a subject. For many such applications, polymeric coatings must be as thin as possible. Polymeric materials for use in delivering an active agent may also be in various three-dimensional shapes.

Conventional active agent delivery systems suffer from limitations that include structural failure due to cracking and delamination from the device surface. Furthermore, they tend to be limited in terms of the range of active agents that can be used, the range of amounts of active agents that can be included within a delivery system, and the range of the rates at which the included active agents are delivered therefrom. This is frequently because many conventional systems include a single polymer.

Thus, there is a continuing need for active agent delivery systems with greater versatility and tunability.

### SUMMARY OF THE INVENTION

The present invention provides active agent delivery systems that have generally good versatility and tunability in controlling the delivery of active agents. Typically, such advantages result from the use of a blend of two or more miscible polymers. These delivery systems can be incorporated into medical devices, e.g., stents, stent grafts, anastomotic connectors, if desired.

The active agent delivery systems of the present invention typically include a blend of at least two miscible polymers, wherein at least one polymer (preferably one of the miscible polymers) is matched to the solubility of the active agent such that the delivery of the active agent preferably occurs predominantly under permeation control. In this context, “predominantly” with respect to permeation control means that at least 50%, preferably at least 75%, and more preferably at least 90%, of the total active agent load is delivered by permeation control.

Permeation control is typically important in delivering an active agent from systems in which the active agent passes through a miscible polymer blend having a “critical” dimension on a micron-scale level (i.e., the net diffusion path is no greater than about 1000 micrometers, although for shaped objects it can be up to about 10,000 microns). Furthermore, it is generally desirable to select polymers for a particular active agent that provide desirable mechanical properties without being detrimentally affected by nonuniform incorporation of the active agent.

In one preferred embodiment, the present invention provides an active agent delivery system that includes an active agent and a hydrophilic miscible polymer blend that includes a hydrophilic polymer (preferably, a polyurethane) and a second polymer having a different swellability in water at 37° C. Preferably, the swellability of the miscible polymer blend controls the delivery of the active agent.

In another preferred embodiment, the present invention provides an active agent delivery system that includes an active agent and a hydrophilic miscible polymer blend that includes a hydrophilic polyurethane and a second polymer having a different swellability in water at 37° C., wherein: the active agent is hydrophilic and has a molecular weight of greater than about 1200 grams per mole (g/mol); the active agent has a solubility parameter, the hydrophilic polyurethane has a hard segment solubility parameter and a soft segment solubility parameter, and the second polymer has at least one solubility parameter; the difference between the solubility parameter of the active agent and the solubility parameter of the hydrophilic polyurethane hard segment is no greater than (i.e., less than or equal to) about 10 J^{1/2}/cm^{3/2} (preferably, no greater than about 5 J^{1/2}/cm^{3/2}, and more preferably, no greater than about 3 J^{1/2}/cm^{3/2}), and/or the difference between the solubility parameter of the active agent and the solubility parameter of the hydrophilic polyurethane soft segment is no greater than about 10 J^{1/2}/cm^{3/2} (preferably, no greater than about 5 J^{1/2}/cm^{3/2}, and more preferably, no greater than about 3 J^{1/2}/cm^{3/2}), and the difference between the solubility parameter of the active agent and at least one solubility parameter of the second polymer (which, if the second polymer is a segmented polymer, is the solubility parameter of the hard and/or soft segment, for example) is no greater than about 10 J^{1/2}/cm^{3/2} (preferably, no greater than about 5 J^{1/2}/cm^{3/2}, and more preferably, no greater than about 3 J^{1/2}/cm^{3/2}); and the difference between the solubility parameter of the hydrophilic polyurethane hard segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}), and/or the difference between the solubility parameter of the hydrophilic polyurethane soft segment and at least one solubility parameter of the second polymer (which, if the second polymer is a segmented polymer, is the solubility parameter of the hard and/or soft segment, for example) is no greater than about 5 J^{1/2}/cm^{3/2}(preferably, no greater than about 3 J^{1/2}/cm^{3/2}). Preferably, the swellability of the miscible polymer blend controls the delivery of the active agent.

As used herein, a “segmented polymer” is composed of multiple blocks, each of which can separate into the phase that is primarily composed of itself. As used herein, a “hard” segment or “hard” phase of a polymer is one that is either crystalline at use temperature or amorphous with a glass transition temperature above use temperature (i.e., glassy), and a “soft” segment or “soft” phase of a polymer is one that is amorphous with a glass transition temperature below use temperature (i.e., rubbery). Herein, a “segment” refers to the chemical formulation and “phase” refers to the morphology, which primarily includes the corresponding segment (e.g., hard segments form a hard phase), but can include some of the other segment (e.g., soft segments in a hard phase).

When referring to the solubility parameter of a segmented polymer, “segment” is used and when referring to Tg of a segmented polymer, “phase” is used. Thus, the solubility parameter, which is typically a calculated value for segmented polymers, refers to the hard and/or soft segment of an individual polymer molecule, whereas the Tg, which is typically a measured value, refers to the hard and/or soft phase of the bulk polymer.

The present invention also provides medical devices that include such active agent delivery systems.

In one preferred embodiment, a medical device is provided that includes: a substrate surface; a polymeric undercoat layer adhered to the substrate surface; and a polymeric top coat layer adhered to the polymeric undercoat layer; wherein the polymeric top coat layer includes an active agent incorporated within a hydrophilic miscible polymer blend that includes a hydrophilic polymer (preferably, a polyurethane) and a second polymer having a different swellability in water at 37° C. (i.e., a swellability different than the swellability of the first polymer). Preferably, the swellability of the miscible polymer blend controls the delivery of the active agent.

In another preferred embodiment, a stent is provided that includes: a substrate surface; a polymeric undercoat layer adhered to the substrate surface; and a polymeric top coat layer adhered to the undercoat layer; wherein the polymeric top coat layer includes an active agent incorporated within a hydrophilic miscible polymer blend that includes a hydrophilic polyurethane and a second polymer having a different swellability in water at 37° C. Preferably, the swellability of the miscible polymer blend controls the delivery of the active agent.

The present invention also provides methods for making an active agent delivery system and delivering an active agent to a subject.

In one embodiment, a method of delivery includes: providing an active agent delivery system including an active agent and a hydrophilic miscible polymer blend comprising a hydrophilic polymer (preferably, a polyurethane) and a second polymer having a different swellability in water at 37° C.; and contacting the active agent delivery system with a bodily fluid, organ, or tissue of a subject. Preferably, the swellability of the miscible polymer blend controls the delivery of the active agent.

In another embodiment, a method of forming an active agent delivery system includes: combining a hydrophilic polymer (preferably, a polyurethane) and a second polymer having a different swellability in water at 37° C. to form a hydrophilic miscible polymer blend; and combining at least one active agent with the miscible polymer blend. Preferably, the swellability of the miscible polymer blend controls the delivery of the active agent.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWING

FIG. 1 is a graph of the delivery of Resten NG from a blend of a hydrophilic polyurethane and a poly(vinyl acetate-co-vinyl pyrrolidone).

FIG. 2 is a graph of the DSC curves of TECOPHILIC HP-60D-60/PVP-VA blends.

FIG. 3 is a graph of the swelling percentage of TECOPHILIC HP-60D-60/PVP-VA blends as a function of PVP-VA content.

### DETAILED DESCRIPTION OF ILLUSTRATIVE EMBODIMENTS

The present invention provides active agent delivery systems that include an active agent for delivery to a subject and a miscible polymer blend. The delivery systems can include a variety of polymers as long as at least two are miscible as defined herein. The active agent may be incorporated within the miscible polymer blend such that it is dissoluted from the blend, or the blend can initially function as a barrier to the environment through which the active agent passes.

Miscible polymer blends are advantageous because they can provide greater versatility and tunability for a greater range of active agents than can conventional systems that include immiscible mixtures or only a single polymer, for example. That is, using two or more polymers, at least two of which are miscible, can generally provide a more versatile active agent delivery system than a delivery system with only one of the polymers. A greater range of types of active agents can typically be used. A greater range of amounts of an active agent can typically be incorporated into and delivered from (preferably, predominantly under permeation control) the delivery systems of the present invention. A greater range of delivery rates for an active agent can typically be provided by the delivery systems of the present invention. At least in part, this is because of the use of a miscible polymer blend that includes at least two miscible polymers. It should be understood that, although the description herein refers to two polymers, the invention encompasses systems that include more than two polymers, as long as a miscible polymer blend is formed that includes at least two miscible polymers.

A miscible polymer blend of the present invention has a sufficient amount of at least two miscible polymers to form a continuous portion, which helps tune the rate of release of the active agent. Such a continuous portion (i.e., continuous phase) can be identified microscopically or by selective solvent etching. Preferably, the at least two miscible polymers form at least 50 percent by volume of a miscible polymer blend.

A miscible polymer blend can also optionally include a dispersed (i.e., discontinuous) immiscible portion. If both continuous and dispersed portions are present, the active agent can be incorporated within either portion. Preferably, the active agent is loaded into the continuous portion to provide delivery of the active agent predominantly under permeation control. To load the active agent, the solubility parameters of the active agent and the portion of the miscible polymer blend a majority of the active agent is loaded into are matched (typically to within no greater than about 10 J^{1/2}/cm^{3/2}, preferably, no greater than about 5 J^{1/2}/cm^{3/2}, and more preferably, no greater than about 3 J^{1/2}/cm^{3/2}). The continuous phase controls the release of the active agent regardless of where the active agent is loaded.

A miscible polymer blend, as used herein, encompasses a number of completely miscible blends of two or more polymers as well as partially miscible blends of two or more polymers. A completely miscible polymer blend will ideally have a single glass transition temperature (Tg), preferably one in each phase (typically a hard phase and a soft phase) of a segmented polymer, due to mixing at the molecular level over the entire concentration range. Partially miscible polymer blends may have multiple Tg's, which can be in one or both of the hard phase and the soft phase for segmented polymers, because mixing at the molecular level is limited to only parts of the entire concentration range. These partially miscible blends are included within the scope of the term “miscible polymer blend” as long as the absolute value of the difference in at least one Tg (Tg_{polymer} 1-Tg_{polymer} 2) for each of at least two polymers within the blend is reduced by the act of blending. The Tg's are measured when the polymers and blends are in the dry state (i.e., when not swollen in water). Tg's can be determined by measuring the mechanical properties, thermal properties, electric properties, etc. as a function of temperature.

A miscible polymer blend can also be determined based on its optical properties. A completely miscible blend forms a stable and homogeneous domain that is transparent, whereas an immiscible blend forms a heterogeneous domain that scatters light and visually appears turbid unless the components have identical refractive indices. Additionally, a phase-separated structure of immiscible blends can be directly observed with microscopy. A simple method used in the present invention to check the miscibility involves mixing the polymers and forming a thin film of about 10 micrometers to about 50 micrometers thick. If such a film is generally as clear and transparent as the least clear and transparent film of the same thickness of the individual polymers prior to blending, then the polymers are completely miscible.

Miscibility between polymers depends on the interactions between them and their molecular structures and molecular weights. The interaction between polymers can be characterized by the so-called Flory-Huggins parameter (χ). When χ is close to zero (0) or even is negative, the polymers are very likely miscible. Theoretically, χ can be estimated from the solubility parameters of the polymers, i.e., % is proportional to the squared difference between them. Therefore, the miscibility of polymers can be approximately predicted. For example, the closer the solubility parameters of the two polymers are the higher the possibility that the two polymers are miscible. Miscibility between polymers tends to decrease as their molecular weights increases.

Thus, in addition to the experimental determinations, the miscibility between polymers can be predicted simply based on the Flory-Huggins interaction parameters, or even more simply, based the solubility parameters of the components. However, because of the molecular weight effect, close solubility parameters do not necessarily guarantee miscibility.

It should be understood that a mixture of polymers needs only to meet one of the definitions provided herein to be miscible. Furthermore, a mixture of polymers may become a miscible blend upon incorporation of an active agent. As used herein, a “hard” phase of a blend includes primarily a segmented polymer's hard segment and optionally at least part of a second polymer blended therein. Similarly, a “soft” phase of a blend includes predominantly a segmented polymer's soft segment and optionally at least part of a second polymer blended therein. Preferably, miscible blends of polymers of the present invention include blends of segmented polymers' soft segments.

The types and amounts of polymers and active agents are typically selected to form a system having a preselected dissolution time (or rate) through a preselected critical dimension of the miscible polymer blend. Swellabilities and solubility parameters of the polymers can be used in guiding one of skill in the art to select an appropriate combination of components in an active agent delivery system, whether the active agent is incorporated into the miscible polymer blend or not. Solubility parameters are generally useful for determining miscibility of the polymers and matching the solubility of the active agent to that of the miscible polymer blend. Swellabilities are generally useful for tuning the dissolution time (or rate) of the active agent. These concepts are discussed in greater detail below.

A miscible polymer blend can be used in combination with an active agent in the delivery systems of the present invention in a variety of formats as long as the miscible polymer blend controls the delivery of the active agent. Preferably, the swellability of the miscible polymer blend (as opposed to the active agent) controls the delivery of the active agent.

In one embodiment, a miscible polymer blend has an active agent incorporated therein. Preferably, such an active agent is dissoluted predominantly under permeation control, which requires at least some solubility of the active agent in the continuous portion (i.e., the miscible portion) of the polymer blend, whether the majority of the active agent is loaded in the continuous portion or not. Dispersions are acceptable as long as little or no porosity channeling occurs during dissolution of the active agent and the size of the dispersed domains is much smaller than the critical dimension of the blends, and the physical properties are generally uniform throughout the composition for desirable mechanical performance. This embodiment is often referred to as a “matrix” system.

In another embodiment, a miscible polymer blend initially provides a barrier to permeation of an active agent. This embodiment is often referred to as a “reservoir” system. A reservoir system can be in many formats with two or more layers. For example, a miscible polymer blend can form an outer layer over an inner layer of another material (referred to herein as the inner matrix material). In another example, a reservoir system can be in the form of a core-shell, wherein the miscible polymer blend forms the shell around the core matrix (i.e., the inner matrix material). At least initially upon formation, the miscible polymer blend in the shell or outer layer could be substantially free of active agent. Subsequently, the active agent permeates from the inner matrix and through the miscible polymer blend for delivery to the subject. The inner matrix material can include a wide variety of conventional materials used in the delivery of active agents. These include, for example, an organic polymer such as those described herein for use in the miscible polymer blends, or a wax, or a different miscible polymer blend. Alternatively, the inner matrix material can be the active agent itself.

For a reservoir system, the release rate of the active agent can be tuned with selection of the material of the outer layer. The inner matrix can include an immiscible mixture of polymers or it can be a homopolymer if the outer layer is a miscible blend of polymers.

As with matrix systems, an active agent in a reservoir system is preferably dissoluted predominantly under permeation control through the miscible polymer blend of the barrier layer (i.e., the barrier polymer blend), which requires at least some solubility of the active agent in the barrier polymer blend. Again, dispersions are acceptable as long as little or no porosity channeling occurs in the barrier polymer blend during dissolution of the active agent and the size of the dispersed domains is much smaller than the critical dimension of the blends, and the physical properties are generally uniform throughout the barrier polymer blend for desirable mechanical performance. Although these considerations may also be desirable for the inner matrix, they are not necessary requirements.

Typically, the amount of active agent within an active agent delivery system of the present invention is determined by the amount to be delivered and the time period over which it is to be delivered. Other factors can also contribute to the level of active agent present, including, for example, the ability of the composition to form a uniform film on a substrate.

Preferably, for a matrix system, an active agent is present within (i.e., incorporated within) a miscible polymer blend in an amount of at least about 0.1 weight percent (wt-%), more preferably, at least about 1 wt-%, and even more preferably, at least about 5 wt-%, based on the total weight of the miscible polymer blend and the active agent. Preferably, for a matrix system, an active agent is present within a miscible polymer blend in an amount of no greater than about 80 wt-%, more preferably, no greater than about 50 wt-%, and most preferably, no greater than about 30 wt-%, based on the total weight of the miscible polymer blend and the active agent. Typically and preferably, the amount of active agent will be at or below its solubility limit in the miscible polymer blend.

Preferably, for a reservoir system, an active agent is present within an inner matrix in an amount of at least about 0.1 wt-%, more preferably, at least about 10 wt-%, and even more preferably, at least about 25 wt-%, based on the total weight of the inner matrix (including the active agent). Preferably, for a reservoir system, an active agent is present within an inner matrix in an amount up to 100 wt-%, and more preferably, no greater than about 80 wt-%, based on the total weight of the inner matrix (including the active agent).

In the active agent delivery systems of the present invention, an active agent is dissolutable through a miscible polymer blend. Dissolution is preferably controlled predominantly by permeation of the active agent through the miscible polymer blend. That is, the active agent initially dissolves into the miscible polymer blend and then diffuses through the miscible polymer blend predominantly under permeation control. Thus, as stated above, for certain preferred embodiments, the active agent is at or below the solubility limit of the miscible polymer blend. Although not wishing to be bound by theory, it is believed that because of this mechanism the active agent delivery systems of the present invention have a significant level of tunability.

If the active agent exceeds the solubility of the miscible polymer blend and the amount of insoluble active agent exceeds the percolation limit, then the active agent could be dissoluted predominantly through a porosity mechanism. In addition, if the largest dimension of the active agent insoluble phase (e.g., particles or aggregates of particles) is on the same order as the critical dimension of the miscible polymer blend, then the active agent could be dissoluted predominantly through a porosity mechanism. Dissolution by porosity control is typically undesirable because it does not provide effective predictability and controllability.

Because the active agent delivery systems of the present invention preferably have a critical dimension on the micron-scale level, it can be difficult to include a sufficient amount of active agent and avoid delivery by a porosity mechanism. Thus, the solubility parameters of the active agent and at least one polymer of the miscible polymer blend are matched to maximize the level of loading while decreasing the tendency for delivery by a porosity mechanism.

One can determine if there is a permeation-controlled release mechanism by examining a dissolution profile of the amount of active agent released versus time (t). For permeation-controlled release from a matrix system, the profile is directly proportional to t^{1/2}. For permeation-controlled release from a reservoir system, the profile is directly proportional to t. Alternatively, under sink conditions (i.e., conditions under which there are no rate-limiting barriers between the polymer blend and the media into which the active agent is dissoluted), porosity-controlled dissolution could result in a burst effect (i.e., an initial very rapid release of active agent).

The active agent delivery systems of the present invention, whether in the form of a matrix system or a reservoir system, for example, without limitation, can be in the form of coatings on substrates (e.g., open or closed cell foams, woven or nonwoven materials), films (which can be free-standing as in a patch, for example), shaped objects (e.g., microspheres, beads, rods, fibers, or other shaped objects), wound packing materials, etc.

As used herein, an “active agent” is one that produces a local or systemic effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject. The term “subject” used herein is taken to include humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, coumarin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylpred nisolone, triamcinolone, betamethasone, dexamethasone, beclomethasone, aclomethasone, amcinonide, clebethasol and clocortolone. Preferably, the active agent is not heparin.

For preferred active agent delivery systems of the present invention, the active agent is typically matched to the solubility of the miscible portion of the polymer blend. For the present invention, at least one polymer of the polymer blend is hydrophilic. Thus, preferred active agents for the present invention are hydrophilic. Preferably, if the active agent is hydrophobic, then at least one of the miscible polymers is hydrophobic, and if the active agent is hydrophilic, then at least one of the miscible polymers is hydrophilic, although this is not necessarily required.

As used herein, in this context (in the context of the polymer of the blend), the term “hydrophilic” refers to a material that will increase in volume by more than 10% or in weight by more than 10%, whichever comes first, when swollen by water at body temperature (i.e., about 37° C.). In contrast, the term “hydrophobic” refers to a material that will not increase in volume by more than 10% or in weight by more than 10%, whichever comes first, when swollen by water at body temperature (i.e., about 37° C.).

As used herein, in this context (in the context of the active agent), the term “hydrophilic” refers to an active agent that has a solubility in water at room temperature (i.e., about 25° C.) of more than 200 micrograms per milliliter. In contrast, the term “hydrophobic” refers to an active agent that has a solubility in water at room temperature (i.e., about 25° C.) of no more than (i.e., less than or equal to) 200 micrograms per milliliter.

For delivery systems in which the active agent is hydrophobic, regardless of the molecular weight, polymers are typically selected such that the molar average solubility parameter of the miscible polymer blend is no greater than 28 J^{1/2}/cm^{3/2} (preferably, no greater than 25 J^{1/2}/cm^{3/2}). For delivery systems in which the active agent is hydrophilic, regardless of the molecular weight, polymers are typically selected such that the molar average solubility parameter of the miscible polymer blend is greater than 21 J^{1/2}/cm^{3/2} (preferably, greater than 25 J^{1/2}/cm^{3/2}). Herein “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

As the size of the active agent gets sufficiently large, diffusion through the polymer is affected. Thus, active agents can be categorized based on molecular weights and polymers can be selected depending on the range of molecular weights of the active agents.

For preferred active agent delivery systems of the present invention, the active agent has a molecular weight of greater than about 1200 g/mol.

Of the active agents listed above, those that are hydrophilic and have a molecular weight of greater than about 1200 g/mol are particularly preferred.

As stated above, the types and amounts of polymers and active agents are typically selected to form a system having a preselected dissolution time (t) through a preselected critical dimension (x) of the miscible polymer blend. This involves selecting at least two polymers to provide a target diffusivity, which is directly proportional to the critical dimension squared divided by the time (x²/t), for a given active agent.

The diffusivity can be easily measured by dissolution analysis using the following equation (see, for example, Kinam Park edited, Controlled Drug Delivery: Challenges and Strategies, American Chemical Society, Washington, D.C., 1997):

$$D = {( \frac{{M}_{t}}{4 {M}_{∞}} )}^{2} \cdot \frac{π {x}^{2}}{t}$$

wherein D=diffusion coefficient; Mₜ=cumulative release; M∞total loading of active agent; x=the critical dimension (e.g., thickness of the film); and t=the dissolution time. This equation is valid during dissolution of up to 60 percent by weight of the initial load of the active agent. Also, blend samples should be in the form of a film.

In refining the selection of the polymers for the desired active agent, the desired delivery time (or rate), and the desired critical dimension, the parameters that can be considered when selecting the polymers for the desired active agent include swellabilities of the polymers, solubility parameters of the polymers, and solubility parameters of the active agents. These can be used in guiding one of skill in the art to select an appropriate combination of components in an active agent delivery system, whether the active agent is incorporated into the miscible polymer blend or not.

For enhancing the tunability of a permeation-controlled delivery system, for example, preferably the polymers are selected such that the difference between the swellabilities in water at 37° C. of at least two of the polymers of the blend is sufficient to provide the target diffusivity. The target diffusivity is determined by the preselected dissolution time (t) for delivery and the preselected critical dimension (x) of the polymer composition and is directly proportional to x²/t.

For enhancing the versatility of a permeation-controlled delivery system, for example, preferably the polymers are selected such that at least one of the following relationships is true: (1) the difference between the solubility parameter of the active agent and at least one solubility parameter of at least one polymer is no greater than about 10 J^{1/2}/cm^{3/2}(preferably, no greater than about 5 J^{1/2}/cm^{3/2}, and more preferably, no greater than about 3 J^{1/2}/cm^{3/2}); and (2) the difference between at least one solubility parameter of each at least two polymers is no greater than about 5 J^{1/2}/cm^{3/2} (preferably, no greater than about 3 J^{1/2}/cm^{3/2}). More preferably, both relationships are true. Most preferably, both relationships are true for all polymers of the blend.

Typically, a compound has only one solubility parameter, although certain polymers, such as segmented copolymers and block copolymers, for example, can have more than one solubility parameter. Solubility parameters can be measured or they are calculated using an average of the values calculated using the Hoy Method and the Hoftyzer-van Krevelen Method (chemical group contribution methods), as disclosed in D. W. van Krevelen, Properties of Polymers, 3^{rd} Edition, Elsevier, Amsterdam. To calculate these values, the volume of each chemical is needed, which can be calculated using the Fedors Method, disclosed in the same reference.

Solubility parameters can also be calculated with computer simulations, for example, molecular dynamics simulation and Monte Carlo simulation. Specifically, the molecular dynamics simulation can be conducted with Accelrys Materials Studio, Accelrys Inc., San Diego, Calif. The computer simulations can be used to directly calculate the Flory-Huggins parameter.

A hydrophilic miscible polymer blend of the present invention includes a hydrophilic polymer, which can be a homopolymer or copolymer. Herein, a “copolymer” includes two or more different repeat units, thereby encompassing terpolymers, tetrapolymers, and the like.

At least one of the polymers of the miscible polymer blend is hydrophilic, and preferably all polymers of the blend are hydrophilic. If one or more of the polymers in the blend is not hydrophilic, the overall blend is preferably hydrophilic.

As used herein in this context (in the context of the individual polymers or blends thereof), the term “hydrophilic” refers to a material (individual polymer or blend) that will increase in volume by at least 10% or in weight by at least 10%, whichever comes first, when swollen by water at body temperature (i.e., about 37° C.). In contrast, the term “hydrophobic” refers to a material that will not increase in volume by more than 10% or in weight by more than 10%, whichever comes first, when swollen by water at body temperature (i.e., about 37° C.). Preferably, particularly for a miscible polymer blend, the term “hydrophilic” refers to a material that will not increase in volume by more than 300% when swollen by water at body temperature (i.e., about 37° C.). Thus, the blends of the present invention are typically not considered hydrogels.

Preferably, all polymers of the miscible polymer blend of the present invention are generally insoluble in water at use temperatures (e.g., body temperature or about 37° C.). However, after the active agent is delivered, one or more of the polymers can dissolve as long as the mechanical integrity of the composition is not sacrificed significantly. In this context, a polymer is insoluble if its mechanical properties are generally maintained while immersed in water at use temperature for at least a period of time generally equivalent to the intended application time.

A miscible polymer blend of the present invention includes at least one polymer that is hydrophilic and a second polymer that has a different swellability in water than the swellability in water of the hydrophilic polymer (the first polymer). The second polymer may be hydrophobic, hydrophilic, or amphiphilic. Generally, if the active agent is hydrophobic, then at least one of the miscible polymers is hydrophobic, and if the active agent is hydrophilic, then at least one of the miscible polymers is hydrophilic.

Preferably, the second polymer is hydrophilic or hydrophobic, and more preferably, the second polymer is hydrophilic. This second polymer may also be a homopolymer or a copolymer. If the polymer is amphiphilic, it is a copolymer or a partially hydrophilically (or hydrophobically) modified homopolymer.

Preferably, the second polymer is a hydrophilic polymer having a swellability in water at 37° C. lower than the swellability in water of the first hydrophilic polymer (e.g., the hydrophilic polyurethane). Thus, the second polymer is preferably selected to decrease the swelling volume ratio of the blend, thereby tuning the diffusivity of the system. The swelling volume ratio is the volume of the polymer swollen with water divided by the volume of the dry polymer.

For example, a preferred combination includes a polyvinyl pyrollidone-co-vinyl acetate copolymer, which has a swellability of greater than 100% (i.e., it is water soluble), and poly(ether urethane), which has a swellability of 60%.

Swellabilities of polymers in water can be easily determined. It should be understood, however, that the swellability results from incorporation of water and not from an elevation in temperature.

Typically, by selecting relatively low and high swell polymers that are miscible, the dissolution kinetics of the system can be tuned. This is advantageous because the range of miscible blends can be used to encompass very different dissolution rates for active agents of similar solubility.

Preferably, higher molecular weights of polymers are desirable for better mechanical properties; however, the molecular weights should not be so high such that the polymer is not soluble in a processing solvent for preferred solvent-coating techniques or not miscible with the other polymer(s) in the blend.

A preferred hydrophilic polymer has a number average molecular weight of at least about 20,000 grams/mole (g/mol), and more preferably at least about 50,000 g/mol. A preferred hydrophilic polymer has a number average molecular weight of no greater than about 10,000,000 g/mol, and more preferably no greater than about 1,000,000 g/mol.

A preferred second polymer, whether it is hydrophilic or hydrophobic, has a number average molecular weight of at least about 10,000 g/mol, and more preferably at least about 80,000 g/mol. A preferred hydrophilic polymer, whether it is hydrophilic or hydrophobic, has a number average molecular weight of no greater than about 10,000,000 g/mol, and more preferably no greater than about 1,000,000 g/mol, and even more preferably no greater than about 300,000 g/mol.

Any one polymer is preferably present in the miscible polymer blend in an amount of at least about 0.1 wt-%, and more preferably up to about 99.9 wt-%, based on the total weight of the blend, depending on the active agent and specific choice of polymers.

Suitable hydrophilic polymers can be naturally occurring or synthetic. They can include, polypeptides (e.g., proteins, oligopeptides) and polynucleotides (e.g., oligonucleotides, DNA, RNA, and analogs thereof. Examples of suitable hydrophilic polymers include, but are not limited to, polyurethanes, polyvinyl alcohols, poly(alkylene ether)s such as polypropylene oxide, polyethylene oxide, and polytetramethyl oxide, polyvinyl pyridines, polyvinyl pyrrolidones, polyacrylonitriles (at least partially hydrolyzed), polyacrylamides, polyvinyl pyrrolidone/polyvinyl acetate copolymers, sulfonated polystyrenes, polyvinyl pyrrolidone/polystyrene copolymers, polysaccharides such as dextran and mucopolysaccharides, xanthan, hydrophilic cellulose derivatives such as hydroxypropyl cellulose and methyl cellulose, hyaluronic acid, hydrophilic polyacrylates and methacrylates such as polyacrylic acid, polymethacrylic acid, and polyhydroxyethyl methacrylate, DNA and RNA or analogs thereof, heparin, chitosan, polyethylene imine, polyacrylamide, as well as other nitrogen-containing polymers (e.g., amine-containing polymers), and combinations thereof. In this context, “combination” means mixtures and copolymers thereof. The mixtures and copolymers can include one or more members of the group and/or other monomers/polymers.

For certain embodiments, the hydrophilic polymer is preferably selected from the group consisting of polyvinyl pyrrolidone, polyvinyl alcohol, polypropylene oxide, polyethylene oxide, polystyrene sulfonate, heparin, chitosan, polyethylene imine, polyacrylamide, and combinations thereof. Examples of copolymers include polyvinyl pyrollidone-co-vinyl acetate copolymer and polyvinyl pyrollidone-styrene copolymer.

For certain other embodiments, the hydrophilic polymer is preferably a hydrophilic polyurethane. A preferred hydrophilic polyurethane includes soft segments having therein polyethylene oxide units. Examples of suitable hydrophilic polyurethanes are poly(ether urethanes) available from Thermedics, Inc. (Woburn, Mass.), under the tradename TECOPHILIC.

Examples of suitable hydrophobic polymers include polyurethanes, polycarbonates, polysulfones, polyphenylene oxides, polyimides, polyamides, polyesters, polyethers, polyketones, polyepoxides, styrene-acrylonitrile copolymers, polyvinyl alkylates, polyvinyl alkyl ethers, polyvinyl acetals, hydrophobic cellulose derivatives such as methyl cellulose, ethyl cellulose, hydroxy propyl cellulose, cellulose acetate, cellulose propionate, cellulose butyrate, cellulose nitrate, hydroxypropyl methyl cellulose, hydroxypropyl ethyl cellulose, methyl ethyl cellulose, cellulose acetate propionate, cellulose acetate butyrate, cellulose propionate butyrate, cellulose acetate propionate butyrate, and combinations thereof. In this context, “combinations” refers to mixtures and copolymers thereof. The copolymers can include one or more members of the group and/or other monomers/polymers.

For certain embodiments, a preferred hydrophobic polymer is a polyurethane. Suitable hydrophobic polyurethanes are available from a variety of sources such as Thermedics, Inc., Woburn, Mass., including polymers marketed under the tradenames TECOPLAST, TECOTHANE, CARBOTHANE, and TECOFLEX. Other preferred polymers include the PELLETHANE and ISOPLAST series available from Dow Chemical Co. (Midland, Mich.), especially PELLETHANE 75D; ELASTHANE, PURSIL, CARBOSIL, BIONATE, and BIOSPAN, available from the Polymer Technology Group, Inc. (Berkeley, Calif.); ESTANE, available from Noveon, Inc. (Cleveland, Ohio); ELAST-EON, available from AorTech Biomaterials (Sidney, Australia); and TEXIN, available from Bayer (Pittsburg, Pa.).

Examples of such polyurethanes include poly(carbonate urethane), poly(ether urethane), poly(ester urethane), poly(siloxane urethane), poly(hydrocarbon urethane), such as those exemplified in U.S. Pat. No. 4,873,308, sulfur-containing polyurethanes, such as those exemplified in U.S. Pat. Nos. 6,149,678, 6,111,052, 5,986,034, end-group modified polyurethanes, such as those commercially available from Polymer Technology Group, Inc., under the trade designation SME, or combinations thereof. Additionally, the polyurethanes may be derived from isocyanates including aromatic and/or aliphatic groups. A particularly preferred polyurethane is a poly(carbonate urethane) or a poly(ether urethane).

Preferably, miscible blends of the present invention include a polyurethane, whether it be hydrophobic or hydrophilic.

Preferably, the difference between the solubility parameter of the active agent and at least one solubility parameter of at least one polymer of the miscible polymer blend is no greater than about 10 J^{1/2}/cm^{3/2} (preferably, no greater than about 5 J^{1/2}/cm^{3/2}, and more preferably, no greater than about 3 J^{1/2}/cm^{3/2}). More preferably, for preferred embodiments that include a polyurethane, at least one of the following relationships is true: the difference between the solubility parameter of the active agent and the solubility parameter of the hydrophilic polyurethane hard segment is no greater than about 10 J^{1/2}/cm^{3/2} (preferably, no greater than about 5 J^{1/2}/cm^{3/2}, and more preferably, no greater than about 3 J^{1/2}/cm^{3/2}); the difference between the solubility parameter of the active agent and the solubility parameter of the hydrophilic polyurethane soft segment is no greater than about 10 J^{1/2}/cm^{3/2} (preferably, no greater than about 5 J^{1/2}/cm^{3/2}, and more preferably, no greater than about 3 J^{1/2}/cm^{3/2}); and the difference between the solubility parameter of the active agent and at least one solubility parameter of the second polymer (which, if the second polymer is a segmented polymer, is the solubility parameter of the hard and/or soft segment, for example) is no greater than about 10 J^{1/2}/cm^{3/2} (preferably, no greater than about 5 J^{1/2}/cm^{3/2}, and more preferably, no greater than about 3 J^{1/2}/cm^{3/2}). Most preferably, the solubility parameter of the active agent is within about 10 J^{1/2}/cm^{3/2} (preferably, within about 5 J^{1/2}/cm^{3/2}, and more preferably, within about 3 J^{1/2}/cm^{3/2}) of at least one solubility parameter of each polymer of the blend.

Preferably, the difference between at least one solubility parameter of each of at least two polymers of the miscible polymer blend is no greater than about 5 J^{1/2}/cm^{3/2}(preferably, no greater than about 3 J^{1/2}/cm^{3/2}). More preferably, at least one of the following relationships is true: the difference between the solubility parameter of the hydrophilic polyurethane hard segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}); and the difference between the solubility parameter of the hydrophilic polyurethane soft segment and at least one solubility parameter of the second polymer (which, if the second polymer is a segmented polymer, is the solubility parameter of the hard and/or soft segment, for example) is no greater than about 5 J^{1/2}/cm^{3/2} (preferably, no greater than about 3 J^{1/2}/cm^{3/2}). Most preferably, if two segmented polymers are used, the difference between the solubility parameters of the hard segments is no greater than about 5 J^{1/2}/cm^{3/2} (preferably, no greater than about 3 J^{1/2}/cm^{3/2}), and the difference between the solubility parameters of the soft segments is no greater than about 5 J^{1/2}/cm^{3/2} (preferably, no greater than about 3 J^{1/2}/cm^{3/2}).

The polymers in the miscible polymer blends can be crosslinked or not. Similarly, the blended polymers can be crosslinked or not. Such crosslinking can be carried out by one of skill in the art after blending using standard techniques.

In the active agent systems of the present invention, the active agent passes through a miscible polymer blend having a “critical” dimension. This critical dimension is along the net diffusion path of the active agent and is preferably no greater than about 1000 micrometers (i.e., microns), although for shaped objects it can be up to about 10,000 microns.

For embodiments in which the miscible polymer blends form coatings or free-standing films (both generically referred to herein as “films”), the critical dimension is the thickness of the film and is preferably no greater than about 1000 microns, more preferably no greater than about 500 microns, and most preferably no greater than about 100 microns. A film can be as thin as desired (e.g., 1 nanometer), but are preferably no thinner than about 10 nanometers, more preferably no thinner than about 100 nanometers. Generally, the minimum film thickness is determined by the volume that is needed to hold the required dose of active agent and is typically only limited by the process used to form the materials. For all embodiments herein, the thickness of the film does not have to be constant or uniform. Furthermore, the thickness of the film can be used to tune the duration of time over which the active agent is released.

For embodiments in which the miscible polymer blends form shaped objects (e.g., microspheres, beads, rods, fibers, or other shaped objects), the critical dimension of the object (e.g., the diameter of a microsphere or rod) is preferably no greater than about 10,000 microns, more preferably no greater than about 1000 microns, even more preferably no greater than about 500 microns, and most preferably no greater than about 100 microns. The objects can be as small as desired (e.g., 10 nanometers for the critical dimension). Preferably, the critical dimension is no less than about 100 microns, and more preferably no less than about 500 nanometers.

In one embodiment, the present invention provides a medical device characterized by a substrate surface overlayed with a polymeric top coat layer that includes a miscible polymer blend, preferably with a polymeric undercoat (primer) layer. When the device is in use, the miscible polymer blend is in contact with a bodily fluid, organ, or tissue of a subject.

The invention is not limited by the nature of the medical device; rather, any medical device can include the polymeric coating layer that includes the miscible polymer blend. Thus, as used herein, the term “medical device” refers generally to any device that has surfaces that can, in the ordinary course of their use and operation, contact bodily tissue, organs or fluids such as blood. Examples of medical devices include, without limitation, stents, stent grafts, anastomotic connectors, leads, needles, guide wires, catheters, sensors, surgical instruments, angioplasty balloons, wound drains, shunts, tubing, urethral inserts, pellets, implants, pumps, vascular grafts, valves, pacemakers, and the like. A medical device can be an extracorporeal device, such as a device used during surgery, which includes, for example, a blood oxygenator, blood pump, blood sensor, or tubing used to carry blood, and the like, which contact blood which is then returned to the subject. A medical device can likewise be an implantable device such as a vascular graft, stent, stent graft, anastomotic connector, electrical stimulation lead, heart valve, orthopedic device, catheter, shunt, sensor, replacement device for nucleus pulposus, cochlear or middle ear implant, intraocular lens, and the like. Implantable devices include transcutaneous devices such as drug injection ports and the like.

In general, preferred materials used to fabricate the medical device of the invention are biomaterials. A “biomaterial” is a material that is intended for implantation in the human body and/or contact with bodily fluids, tissues, organs and the like, and that has the physical properties such as strength, elasticity, permeability and flexibility required to function for the intended purpose. For implantable devices in particular, the materials used are preferably biocompatible materials, i.e., materials that are not overly toxic to cells or tissue and do not cause undue harm to the body.

The invention is not limited by the nature of the substrate surface for embodiments in which the miscible polymer blends form polymeric coatings. For example, the substrate surface can be composed of ceramic, glass, metal, polymer, or any combination thereof. In embodiments having a metal substrate surface, the metal is typically iron, nickel, gold, cobalt, copper, chrome, molybdenum, titanium, tantalum, aluminum, silver, platinum, carbon, and alloys thereof. A preferred metal is stainless steel, a nickel titanium alloy, such as NITINOL, or a cobalt chrome alloy, such as NP35N.

A polymeric coating that includes a miscible polymer blend can adhere to a substrate surface by either covalent or non-covalent interactions. Non-covalent interactions include ionic interactions, hydrogen bonding, dipole interactions, hydrophobic interactions and van der Waals interactions, for example.

Preferably, the substrate surface is not activated or functionalized prior to application of the miscible polymer blend coating, although in some embodiments pretreatment of the substrate surface may be desirable to promote adhesion. For example, a polymeric undercoat layer (i.e., primer) can be used to enhance adhesion of the polymeric coating to the substrate surface. Suitable polymeric undercoat layers are disclosed in Applicants' copending U.S. Provisional Application Serial No. 60/403,479, filed on Aug. 13, 2002, and U.S. patent application Ser. No. ______, filed on even date herewith, both entitled MEDICAL DEVICE EXHIBITING IMPROVED ADHESION BETWEEN POLYMERIC COATING AND SUBSTRATE. A particularly preferred undercoat layer disclosed therein consists essentially of a polyurethane. Such a preferred undercoat layer includes a polymer blend that contains polymers other than polyurethane but only in amounts so small that they do not appreciably affect the durometer, durability, adhesive properties, structural integrity and elasticity of the undercoat layer compared to an undercoat layer that is exclusively polyurethane.

When a stent or other vascular prosthesis is implanted into a subject, restenosis is often observed during the period beginning shortly after injury to about four to six months later. Thus, for embodiments of the invention that include stents, the generalized dissolution rates contemplated are such that the active agent should ideally start to be released immediately after the prosthesis is secured to the lumen wall to lessen cell proliferation. The active agent should then continue to dissolute for up to about four to six months in total.

The invention is not limited by the process used to apply the polymer blends to a substrate surface to form a coating. Examples of suitable coating processes include solution processes, powder coating, melt extrusion, or vapor deposition.

A preferred method is solution coating. For solution coating processes, examples of solution processes include spray coating, dip coating, and spin coating. Typical solvents for use in a solution process include tetrahydrofuran (THF), methanol, ethanol, ethylacetate, dimethylformamide (DMF), dimethyacetamide (DMA), dimethylsulfoxide (DMSO), dioxane, N-methylpyrollidone, chloroform, hexane, heptane, cyclohexane, toluene, formic acid, acetic acid, and/or dichloromethane. Single coats or multiple thin coats can be applied.

Similarly, the invention is not limited by the process used to form the miscible polymer blends into shaped objects. Such methods would depend on the type of shaped object. Examples of suitable processes include extrusion, molding, micromachining, emulsion polymerization methods, electrospray methods, etc.

For preferred embodiments in which the active agent delivery system includes one or more coating layers applied to a substrate surface, a preferred embodiment includes the use of a primer, which is preferably applied using a “reflow method,” which is described in Applicants' copending U.S. Provisional Application Serial No. 60/403,479, filed on Aug. 13, 2002, and U.S. patent application serial No. ______, filed on even date herewith, both MEDICAL DEVICE EXHIBITING IMPROVED ADHESION BETWEEN POLYMERIC COATING AND SUBSTRATE.

Preferably, in this “reflow method,” the device fabrication process involves first applying an undercoat polymer to a substrate surface to form the polymeric undercoat layer, followed by treating the polymeric undercoat layer to reflow the undercoat polymer, followed by applying a miscible polymer blend, preferably with an active agent incorporated therein, to the reformed undercoat layer to form a polymeric top coat layer. Reflow of the undercoat polymer can be accomplished in any convenient manner, e.g., thermal treatment, infrared treatment, ultraviolet treatment, microwave treatment, RF treatment, mechanical compression, or solvent treatment. To reflow the undercoat polymer, the undercoat layer is heated to a temperature that is at least as high as the “melt flow temperature” of the undercoat polymer, and for a time sufficient to reflow the polymer. The temperature at which the polymer enters the liquid flow state (i.e., the “melt flow temperature”) is the preferred minimum temperature that is used to reflow the polymer according to the invention. Typically 1 to 10 minutes is the time period used to reflow the polymer using a thermal treatment in accordance with the invention. The melt flow temperature for a polymer is typically above the Tg (the melt temperature for a glass) and the Tm (the melt temperature of a crystal) of the polymer.

### EXAMPLES

The present invention is illustrated by the following examples. It is to be understood that the particular examples, materials, amounts, and procedures are to be interpreted broadly in accordance with the scope and spirit of the invention as set forth herein.

TECOPHILIC HP-60D-60 polyurethane, Thermedics, Inc. Woburn, Mass., and poly(vinyl acetate-co-vinyl pyrrolidone) (PVP-VA), Sigma-Aldrich Chemical Company, Milwaukee, Wis., were the matrix polymers used in this example. RESTEN NG, a 7000 molecular weight, water-soluble antisense oligonuctleotide, AVI Biopoharma, Corvallis, Oreg., was the active agent used in this example. The soft segment of TECOPHILIC polyurethane contains a mixture of poly(ethylene oxide) (PEO) and poly(tetramethylene oxide) (PTMO). The solubility parameter of this soft segment was estimated to be from 19 J^{1/2}/cm^{3/2} (PTMO) to 23 J^{1/2}/cm^{3/2} (PEO) based on Hoftyzer and van Kevelen's (H-vK) method (where the volumes of the chemicals were calculated based on Fedors' method) (Chapter 7, D. W. van Krevelen, Properties of Polymers, 3rd ed., Elsevier, 1990, where Table 7.8 was for Hoftyzer and van Kevelen's method, Table 7.3 for Fedors' method). The solubility parameter of PVP-VA was estimated to be 23 J^{1/2}/cm^{3/2} (molar average over PVP and VA monomers based on their mass ratio in polymer) based on the same method.

TECOPHILIC polyurethane was dissolved in anhydrous chloroform, Sigma-Aldrich Chemical Company, Milwaukee, Wis., at a concentration of 1 wt-% polyurethane. The polyurethane and solvent were combined in a glass vial, which was sealed and shaken until the polyurethane was completely dissolved (by visual observation). Medtronic Model S-670 coronary stents (3.0 mm×18 mm), which had previously been cleaned by ultrasonication in methanol and air dried, were spray coated with 50 to 100 micrograms of the polyurethane coating prepared above. A proprietary spray unit was used to coat the stents in this example, but any spray unit capable of applying a finely atomized mist of the polymer solution to the stent should be adequate. After spray coating with 50 to 100 micrograms of polyurethane solution, the stents were allowed to dry in lab ambient conditions, 25° C. and 15% relative humidity (RH), for four hours. After the stents were dried they were placed in an oven at 220° C. for 20 minutes to reflow the primer coating. After reflow the stents were removed from the oven and allowed to cool to room temperature.

TECOPHILIC polyurethane was dissolved in a solvent blend containing 80 wt-% anhydrous chloroform, Sigma-Aldrich Chemical Company, Milwaukee, Wis., and 20 wt-% anhydrous methanol, Sigma-Aldrich Chemical Company, Milwaukee, Wis. The mixture was shaken until the polymer was completely dissolved (by visual observation). The concentration of TECOPHILIC in the solution was 1 wt-%. This solution is referred to as A.

RESTEN NG oligonuctleotide was dissolved in a solvent blend containing 80 wt-% anhydrous chloroform, Sigma-Aldrich Chemical Company, Milwaukee, Wis., and 20 wt-% anhydrous methanol, Sigma-Aldrich Chemical Company, Milwaukee, Wis. The mixture was shaken until the polymer was completely dissolved (by visual observation). The concentration of RESTEN NG in the solution was 1 wt-%. This solution is referred to as B.

PVP-VA was dissolved in a solvent blend containing 80 wt-% anhydrous chloroform, Sigma-Aldrich Chemical Company, Milwaukee, Wis., and 20 wt-% anhydrous methanol, Sigma-Aldrich Chemical Company, Milwaukee, Wis. The mixture was shaken until the polymer was completely dissolved (by visual observation). The concentration of PVP-VA in the solution was 1 wt-%. This solution is referred to as C.

The solutions A, B, and C were combined as shown in Table 1 below to make solutions with 1% overall “solids” concentration. The “solids” in each solution were comprised of 10 wt-% RESTEN NG and the remainder a blend of TECOPHILIC polyurethane and PVP-VA as denoted in Table 1.

TABLE 1

| | Solution 1: 0% | Solution 2: 10% | Solution 3: 15% |
|---|---|---|---|
| | PVP-VA | PVP-VA | PVP-VA |
| A | 2708 mg | 2290 mg | 2250 mg |
| B | 315 mg | 302 mg | 302 mg |
| C | 0 | 306 mg | 461 mg |

Solutions 1-3 were filtered with a 0.45-micron (microgram) filter and sprayed on the primed stents prepared above. The same proprietary spray unit and process that was used to primed the stent was used to apply the top coat, although any spray unit capable of applying a finely atomized mist of the polymer and drug solution to the stent could have been used. The coated stents were dried at 45° C. in a vacuum oven for 12 hours. Approximately 2000 micrograms (μg) of coating was applied to each stent, and the actual coating weight was used to calculate the theoretical amount of active agent on each stent based on the coating solution formulation.

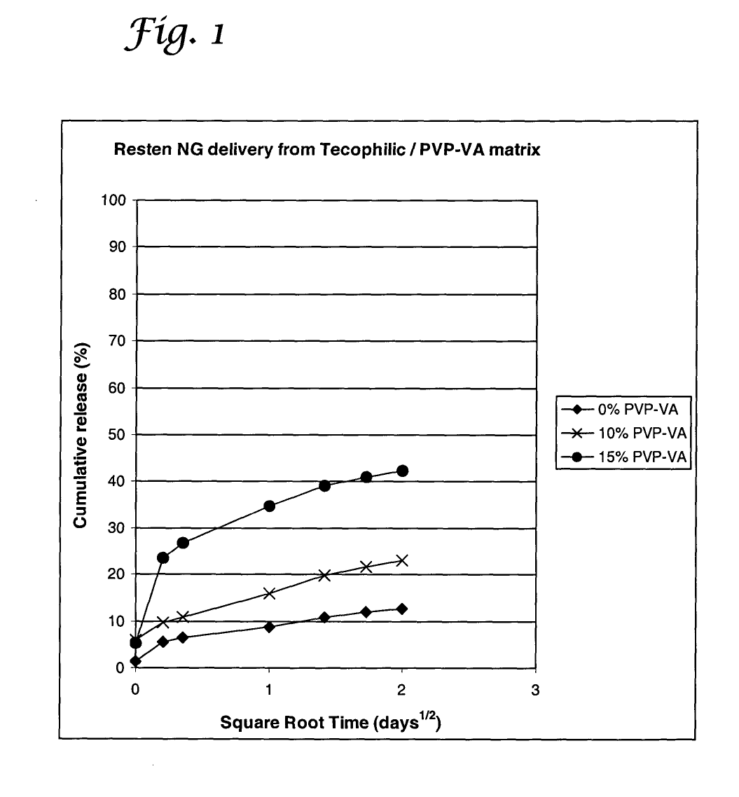

Dissolution testing was conducted on the stents coated above. Each stent was placed in a vial with 3.0 milliliters (mL) of phosphate buffered saline solution (PBS, potassium phosphate monobasic (NF tested), 0.144 grams per liter (g/L), sodium chloride (USP tested), 9 g/L, and sodium phosphate dibasic (USP tested) 0.795 g/L, pH=7.0 to 7.2 at 37° C., purchased from HyClone, Logan, Utah) that was preheated to 37° C. The vials were stored in an incubator-shaker at 37° C. and agitated at about 50 revolutions per minute. At designated times (1 minute, 1 hour, 3 hours, 1 day, 2 days, 3 days, and 4 days in this study) the entire volume of PBS was removed from the sample vial (the vial was quickly refilled with 3.0 mL of fresh PBS that was preheated to 37° C.) and analyzed by UV-VIS Spectrophotometry (HP 4152A) at 260 nanometers (nm). The concentration of RESTEN NG in each sample was determined by comparision to a standard curve. The cumulative amount of RESTEN NG released was divided by the theoretical RESTEN NG load for each stent and plotted against square root time. The results are presented in FIG. 1.

Although there was an initial burst of RESTEN NG released over the first hour, the remainder of the release curve was proportional to square root time indicating the RESTEN NG was released under permeation control. The rate of delivery correlated with the ratio of TECOPHILIC to PVP-VA in the matrix polymer blend. Coatings with more PVP-VA delivered RESTEN NG more quickly.

Miscibility between TECOPHILIC polyurethane and PVP-VA was tested with a PYRIS 1 differential scanning calorimeter (DSC), PerkinElmer Company, Wellesley, Mass. TECOPHILIC polyurethane and PVP-VA were dissolved in the same solvent and in the same way to make about 5 wt-% solutions. The two solutions were mixed at various ratios to make samples with the weight percentages of PVP-VA ranging from 0 to 100%. The blend samples were dried under protection of nitrogen gas. Before doing the test, the samples were further dried under reduced pressure at room temperature. The DSC scans were programmed from −100° C. to 230° C. at 40° C./minute. The samples were scanned twice. The second scan that had less noise were used. The sample size was about 10 milligrams (mg). The same procedure was used for all the Tg determinations in this example.

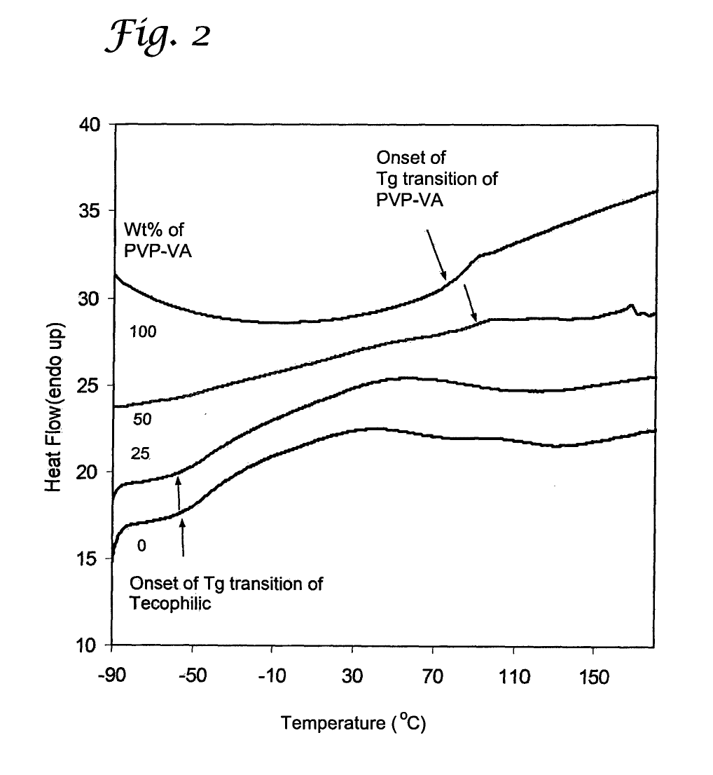

As shown in FIG. 2, the pure TECOPHILIC polyurethane had a glass transition at about −53° C. (onset temperature determined with PYRIS version 5.0 software). This Tg was considered to be associated with the soft domain. The Tg of the hard domain was higher than room temperature because this resin was fairly rigid at room temperature (Durometer 41 D). The pure PVP-VA had a Tg transition at a higher temperature (76° C.). When TECOPHILIC polyurethane was mixed with 20 wt-% of PVP-VA, its DSC curve was essentially not changed; but the Tg of PVP-VA disappeared. When the two polymers were mixed at a ratio of 50/50 by weight, the Tg transition of TECOPHILIC polyurethane disappeared. There was a very weak transition at the temperature around the Tg of PVP-VA. The disappearance of Tg transitions indicated that the two polymers were at least partially miscible.

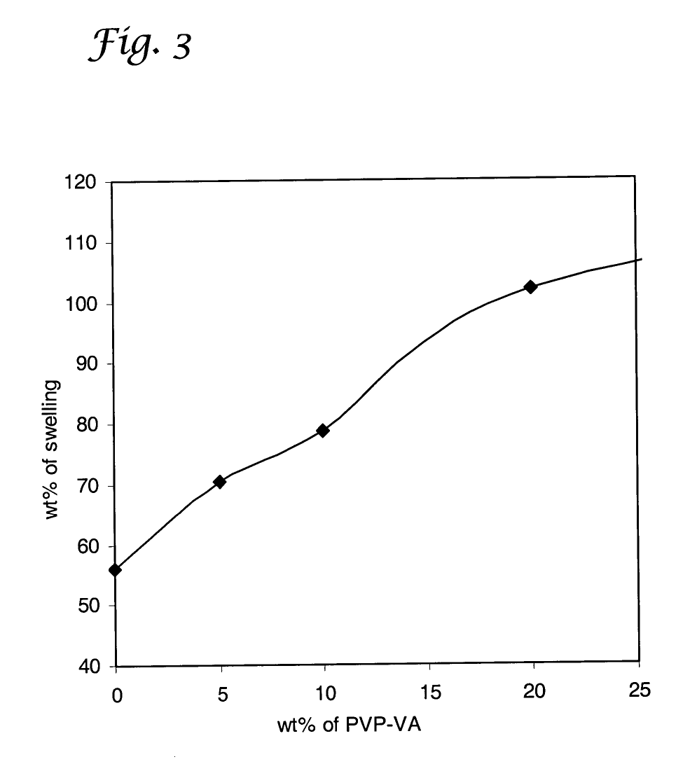

Swelling tests were conducted with the same samples as for the DSC tests. Fully dried samples (Weight 1=50 to 100 mg) were put in a glass vial containing 5 mL of phosphate buffered saline solution (PBS, potassium phosphate monobasic (NF tested), 0.144 g/L, sodium chloride (USP tested), 9 g/L, and sodium phosphate dibasic (USP tested) 0.795 g/L, pH=7.0 to 7.2 at 37° C., purchased from HyClone, Logan, Utah). The vials were stored in an incubator-shaker at 37° C. and agitated at about 50 revolutions per minute for about one day. The samples were taken out of the PBS. A piece of tissue was used to soak the free PBS from sample surfaces. The samples were again weighed (Weight 2). Then, the samples were dried under reduced pressure at room temperature overnight. The samples were weighed for a third time (Weight 3). The swelling percentage was calculated by subtracting Weight 3 from Weight 2 and dividing by Weight 3. Pure TECOPHILIC polyurethane was swollen by about 56%. Pure PVP-VA was completely dissolved in PBS. The swelling percentage was plotted as a function of PVP-VA content in FIG. 3 for the samples containing up to 20-wt % of PVP-VA. This clearly shows that increasing the PVP-VA content from 0 to 20 wt-% increases the swelling ratio of the blends from 56 to 101 wt-%. The weight loss (Weight 1-Weight 3) due to the leaching of PVP-VA into PBS was less than 1 wt-% for the samples containing no more than 10-wt % of PVP-VA.

The complete disclosures of all patents, patent applications including provisional patent applications, and publications, and electronically available material cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

What is claimed is:

1. An active agent delivery system comprising an active agent and a hydrophilic miscible polymer blend comprising a hydrophilic polymer and a second polymer having a different swellability in water at 37° C., wherein the swellability of the miscible polymer blend controls the delivery of the active agent.

2. The system of claim 1 wherein the hydrophilic polymer is a hydrophilic polyurethane.

3. The system of claim 1 wherein the hydrophilic polymer is selected from the group consisting of polyvinyl pyrrolidone, polyvinyl alcohol, polypropylene oxide, polyethylene oxide, polystyrene sulfonate, heparin, chitosan, polyethylene imine, polyacrylamide, and combinations thereof.

4. The system of claim 3 wherein the miscible polymer blend comprises a polyvinyl pyrollidone-co-vinyl acetate copolymer and a poly(ether urethane).

5. The system of claim 1 wherein the second polymer is a hydrophilic polymer or a hydrophobic polymer.

6. The system of claim 5 wherein the second polymer is a hydrophilic polyurethane.

7. The system of claim 6 wherein the hydrophilic polyurethane comprises soft segments comprising polyethylene oxide units.

8. The system of claim 1 wherein the active agent is incorporated within the miscible polymer blend.

9. The system of claim 8 wherein the active agent is present in the miscible polymer blend in an amount of about 0.1 wt-% to about 80 wt-%, based on the total weight of the miscible polymer blend and the active agent.

10. The system of claim 1 wherein miscible polymer blend initially provides a barrier for the active agent.

11. The system of claim 10 wherein the active agent is incorporated within an inner matrix.

12. The system of claim 11 wherein the active agent is present in the inner matrix in an amount of about 0.1 wt-% to about 100 wt-%, based on the total weight of the inner matrix including the active agent.

13. The system of claim 1 wherein:
the active agent has a solubility parameter, the hydrophilic polymer has at least one solubility parameter, and the second polymer has at least one solubility parameter; and
at least one of the following relationships is true:
the difference between the solubility parameter of the active agent and at least one solubility parameter of the hydrophilic polymer is no greater than about 10 J^{1/2}/cm^{3/2}; and
the difference between the solubility parameter of the active agent and at least one solubility parameter of the second polymer is no greater than about 10 J^{1/2}/cm^{3/2}.

14. The system of claim 1 wherein the difference between at least one solubility parameter of the hydrophilic polymer and at least one solubility parameter of the second polymer is no greater than about 5 J^{1/2}/cm^{3/2}.

15. The system of claim 1 wherein:
the hydrophilic polymer has at least one solubility parameter and the second polymer has at least one solubility parameter; and
the difference between at least one solubility parameter of the hydrophilic polymer and at least one solubility parameter of the second polymer is no greater than about 5 J^{1/2}/cm^{3/2}.

16. The system of claim 1 wherein the active agent is hydrophilic and has a molecular weight of greater than about 1200 g/mol.

17. The system of claim 1 wherein the active agent is at least one of a polypeptide or a polynucleotide.

18. The system of claim 1 wherein the hydrophilic polymer is present in the miscible polymer blend in an amount of about 0.1 wt-% to about 99.9 wt-%, based on the total weight of the blend.

19. The system of claim 1 wherein the second polymer is present in the miscible polymer blend in an amount of about 0.1 wt-% to about 99.9 wt-%, based on the total weight of the blend.

20. The system of claim 1 which is in the form of microspheres, beads, rods, fibers, or other shaped objects.

21. The system of claim 20 wherein the critical dimension of the object is no greater than about 10,000 microns.

22. The system of claim 1 which is in the form of a film.

23. The system of claim 22 wherein the thickness of the film is no greater than about 1000 microns.

24. The system of claim 23 wherein the film forms a patch or a coating on a surface.

25. An active agent delivery system comprising an active agent and a hydrophilic miscible polymer blend comprising a first polymer and a second polymer having a different swellability in water at 37° C., wherein:
the active agent is hydrophilic and has a molecular weight of greater than about 1200 g/mol;
the active agent has a solubility parameter, the first polymer has at least one solubility parameter, and the second polymer has at least one solubility parameter;
the difference between the solubility parameter of the active agent and at least one solubility parameter of the first polymer is no greater than about 10 J^{1/2}/cm^{3/2}, and the difference between the solubility parameter of the active agent and at least one solubility parameter of the second polymer is no greater than about 10 J^{1/2}/cm^{3/2};
the difference between at least one solubility parameter of the first polymer and at least one solubility parameter of the second polymer is no greater than about 5 J^{1/2}/cm^{3/2}; and
the swellability of the miscible polymer blend controls the delivery of the active agent.

26. An active agent delivery system comprising an active agent and a hydrophilic miscible polymer blend comprising a hydrophilic polymer and a second polymer having a different swellability in water at 37° C., wherein the swellability of the miscible polymer blend controls the delivery of the active agent, and further wherein delivery of the active agent occurs predominantly under permeation control.

27. A medical device comprising the active agent delivery system of claim 1.

28. A medical device comprising the active agent delivery system of claim 25.

29. A medical device comprising the active agent delivery system of claim 26.

30. A medical device comprising:
a substrate surface;
a polymeric undercoat layer adhered to the substrate surface; and
a polymeric top coat layer adhered to the polymeric undercoat layer; wherein the polymeric top coat layer comprises an active agent incorporated within a hydrophilic miscible polymer blend comprising a hydrophilic polymer and a second polymer having a different swellability in water at 37° C., and further, wherein the swellability of the miscible polymer blend controls the delivery of the active agent

31. The medical device of claim 30 wherein the hydrophilic polymer is a hydrophilic polyurethane.

32. The medical device of claim 30 wherein the hydrophilic polymer is selected from the group consisting of polyvinyl pyrrolidone, polyvinyl alcohol, polypropylene oxide, polyethylene oxide, polystyrene sulfonate, polysaccharide, and combinations thereof.

33. The medical device of claim 30 wherein the second polymer is a hydrophilic polymer or a hydrophobic polymer.

34. The medical device of claim 33 wherein the second polymer is a hydrophilic polyurethane.

35. The medical device of claim 34 wherein the hydrophilic polyurethane comprises soft segments comprising polyethylene oxide units.

36. The medical device of claim 30 which is an implantable device.

37. The medical device of claim 30 which is an extracorporeal device.

38. The medical device of claim 30 selected from the group consisting of a stent, stent graft, anastomotic connector, lead, needle, guide wire, catheter, sensor, surgical instrument, angioplasty balloon, wound drain, shunt, tubing, urethral insert, pellet, implant, blood oxygenator, pump, vascular graft, valve, pacemaker, orthopedic device, replacement device for nucleus pulposus, and intraocular lense.

39. The medical device of claim 30 wherein the active agent is hydrophilic and has a molecular weight of greater than about 1200 g/mol.

40. The medical device wherein delivery of the active agent occurs predominantly under permeation control.

41. A stent comprising:
a substrate surface;
a polymeric undercoat layer adhered to the substrate surface; and
a polymeric top coat layer adhered to the undercoat layer;
wherein the polymeric top coat layer comprises an active agent incorporated within a hydrophilic miscible polymer blend comprising a hydrophilic polyurethane and a second polymer having a different swellability in water at 37° C., and further wherein the swellability of the miscible polymer blend controls the delivery of the active agent.

42. The stent of claim 41 wherein the active agent is hydrophilic and has a molecular weight of greater than about 1200 g/mol.

43. The stent wherein delivery of the active agent occurs predominantly under permeation control.

44. A method for delivering an active agent to a subject, the method comprising:
providing an active agent delivery system comprising an active agent and a hydrophilic miscible polymer blend comprising a hydrophilic polymer and a second polymer having a different swellability in water at 37° C.; and
contacting the active agent delivery system with a bodily fluid, organ, or tissue of a subject;
wherein the swellability of the miscible polymer blend controls the delivery of the active agent.

45. The method of claim 44 wherein the hydrophilic polymer is a hydrophilic polyurethane.

46. The method of claim 44 wherein the hydrophilic polymer is selected from the group consisting of polyvinyl pyrrolidone, polyvinyl alcohol, polypropylene oxide, polyethylene oxide, polystyrene sulfonate, heparin, chitosan, polyethylene imine, polyacrylamide, and combinations thereof.

47. The method of claim 44 wherein the second polymer is a hydrophilic polymer or a hydrophobic polymer.

48. The method of claim 47 wherein the second polymer is a hydrophilic polyurethane.

49. The method of claim 44 wherein the active agent is incorporated within the miscible polymer blend.

50. The method of claim 44 wherein the active agent is incorporated within an inner matrix and the miscible polymer blend initially provides a barrier to permeation of the active agent.

51. The method of claim 44 wherein the active agent is hydrophilic and has a molecular weight of greater than about 1200 g/mol.

52. The method of claim 44 wherein delivery of the active agent occurs predominantly under permeation control.

53. A method of forming an active agent delivery system comprising:
combining a hydrophilic polymer and a second polymer having a different swellability in water at 37° C. to form a hydrophilic miscible polymer blend; and
combining at least one active agent with the miscible polymer blend;
wherein the swellability of the miscible polymer blend controls the delivery of the active agent.

54. The method of claim 53 wherein the hydrophilic polymer is a hydrophilic polyurethane.

55. The method of claim 53 wherein the hydrophilic polymer is selected from the group consisting of polyvinyl pyrrolidone, polyvinyl alcohol, polypropylene oxide, polyethylene oxide, polystyrene sulfonate, heparin, chitosan, polyethylene imine, polyacrylamide, and combinations thereof.

56. The method of claim 53 wherein the second polymer is a hydrophilic polymer or a hydrophobic polymer.

57. The method of claim 56 wherein the second polymer is a hydrophilic polyurethane.

58. The method of claim 53 wherein the active agent is incorporated within the miscible polymer blend.

59. The method of claim 53 wherein the active agent is incorporated within an inner matrix and the miscible polymer blend initially provides a barrier to permeation of the active agent.

60. The method of claim 53 wherein the active agent is hydrophilic and has a molecular weight of greater than about 1200 g/mol.
